(19) United States
(12) Patent Application Publication
(10) Pub. No.: US 2026/0096968 A1
(43) Pub. Date: Apr. 9, 2026

(54) ACTIVE PREMIX CONTAINING ALUMINUM ZIRCONIUM SALTS FOR ANTIPERSPIRANT PRODUCTS

(71) Applicant: THE PROCTER & GAMBLE COMPANY, Cincinnati, OH (US)

(72) Inventors: Wade Charles HENKE, Mason, OH (US); Shawn David MCCONAUGHY, Cincinnati, OH (US); David Frederick SWAILE, Cincinnati, OH (US)

(21) Appl. No.: 19/349,081

(22) Filed: Oct. 3, 2025

Related U.S. Application Data
(60) Provisional application No. 63/702,962, filed on Oct. 3, 2024.
(60) Provisional application No. 63/875,733, filed on Sep. 4, 2025.

(51) Int. Cl. A61K 8/28; A61K 8/02; A61K 8/20; A61K 8/44; A61Q 15/00

(52) U.S. Cl. CPC A61K 8/28; A61K 8/0241; A61K 8/20; A61K 8/44; A61Q 15/00; A61K 2800/412; A61K 2800/58; A61K 2800/5922; A61K 2800/621; A61K 2800/651

(57) ABSTRACT

A particulate active premix composition, and methods of making the premix composition. The premix composition includes an aluminum zirconium salt, an amino acid, and greater than 1% of an alkali metal salt. The composition can be a particle. The particulate active premix can be incorporated into antiperspirant products.

### FIELD OF THE INVENTION

The present disclosure relates to an antiperspirant composition and methods relating thereto. More particularly, the antiperspirant composition is a particle or aqueous solution that includes an aluminum zirconium tri-, tetra-, penta-, and/or octa-chlorohydrate salt, an amino acid, and greater than 1% of an alkali metal salt.

### BACKGROUND OF THE INVENTION

Aluminum-zirconium (Al—Zr) based antiperspirant (AP) salts have gained widespread acceptance in AP products due to their efficacy in reducing sweat production. These salts can be integrated with a variety of other formulation ingredients, including fragrances and moisturizers, further enhancing their appeal in a variety of antiperspirant products including anhydrous sticks, clear gels, soft solids, and roll-on applications.

The primary mechanism of action for Al—Zr salts in antiperspirants involves the temporary occlusion of sweat glands. Upon application, these salts interact with sweat, reacting with moisture and proteins to create a gel-like substance. This gel effectively plugs the sweat ducts, thereby obstructing the flow of sweat to the skin's surface and minimizing perspiration.

While Al—Zr salts are generally regarded as effective ingredients in antiperspirants, many consumers are seeking products that offer even greater efficacy. This can be accomplished by making the Al—Zr salt more hygroscopic, enabling the formation of more effective plugs by facilitating mass transport of the AP active into the sweat duct. However, excessive hygroscopicity can lead to issues in both manufacturing and shipping. If Al—Zr salts are too hygroscopic, they can easily absorb moisture from the air, turning the powder sticky and intractable, and in more extreme cases, into an unmanageable gel or liquid, making it challenging to use these salts in antiperspirant formulations.

Therefore, there is a need for Al—Zr-based AP salts that are optimized for hygroscopicity. Such salts would facilitate the transfer of the AP active salt from the skin's surface into the sweat ducts, leading to more effective plug formation, which is important for antiperspirant effectiveness. Additionally, these salts can maintain the stability and processability required for practical application in manufacturing antiperspirant formulations and shipping of antiperspirant formulations and active salts.

### SUMMARY OF THE INVENTION

A particulate active premix composition comprising: (a) an aluminum zirconium salt chosen from aluminum zirconium trichlorohydrate salt, aluminum zirconium tetrachlorohydrate salt, aluminum zirconium pentachlorohydrate salt, aluminum zirconium octachlorohydrate salt, or mixtures thereof; (b) an amino acid; (c) greater than 1% of an alkali metal salt; wherein the composition comprises a plurality of particles or a solution.

A particulate active premix composition comprising: (a) an aluminum zirconium salt chosen from aluminum zirconium trichlorohydrate salt, aluminum zirconium tetrachlorohydrate salt, aluminum zirconium pentachlorohydrate salt, aluminum zirconium octachlorohydrate salt, or mixtures thereof; (b) an amino acid comprising glycine; (c) greater than 1% of an alkali metal salt comprising sodium chloride; wherein the composition comprises a plurality of particles or a solution.

A method of making a particulate antiperspirant active premix comprising: (a) providing an aqueous solution of an aluminum zirconium salt with a USP assay from about 10% to about 50%; wherein the aluminum zirconium salt is chosen from aluminum zirconium trichlorohydrate salt, aluminum zirconium tetrachlorohydrate salt, aluminum zirconium pentachlorohydrate salt, aluminum zirconium octachlorohydrate salt, or mixtures thereof; (b) adding an alkali metal salt at a concentration of more than 0.5%, by weight of the aqueous solution, and an amino acid to form the aqueous solution premix; (c) after step (b), drying the aqueous premix solution to a plurality of particles, forming the particulate antiperspirant active premix; and (d) optionally grinding the particulate antiperspirant active premix to a Dv50 particle size less than 50 microns.

### BRIEF DESCRIPTION OF THE DRAWINGS

While the specification concludes with claims particularly pointing out and distinctly claiming the subject matter of the present invention, it is believed that the invention can be more readily understood from the following description taken in connection with the accompanying drawings, in which:

FIG. 1 shows the Al:Zr atomic ratio and the Metal:Cl atomic ratio of aluminum zirconium tri-, tetra-, penta-, or octa-chlorohydrate;

FIG. 2A shows the change in mass vs. time for Active Premix Examples 1A (0% NaCl), 1B (5% NaCl), and 1C (10% NaCl);

FIG. 2B shows an image from an SEM microscope of Active Premix Example 1A;

FIG. 2C shows an image from an SEM microscope of Active Premix Example 1B;

FIG. 3A shows the change in mass vs. time for Active Premix Examples 2A (0% NaCl), 2B (5% NaCl), and 2C (10% NaCl);

FIG. 3B shows an image from an SEM microscope of Active Premix Example 2A;

FIG. 3C shows an image from an SEM microscope of Active Premix Example 2B;

FIG. 4A shows the change in mass vs. time for Active Premix Examples 3A (0% NaCl), 3B (5% NaCl), and 3C (10% NaCl);

FIG. 4B shows an image from an SEM microscope of Active Premix Example 3A;

FIG. 4C shows an image from an SEM microscope of Active Premix Example 3B;

FIG. 5A shows the change in mass vs. time for Active Premix Examples 4A (0% NaCl), 1B (5% NaCl), and 1C (10% NaCl);

FIG. 5B shows an image from an SEM microscope of Active Premix Example 4A;

FIG. 5C shows an image from an SEM microscope of Active Premix Example 4B; and FIG. 6 is a representative chromatogram for Peaks I-V.

### DETAILED DESCRIPTION OF THE INVENTION

Aluminum-zirconium (Al—Zr) salts are widely used as active ingredients in antiperspirants (APs). The effectiveness of these salts can be improved through various treatment methods. One such method involves heating the salts prior to spray drying, which helps increase the concentration of smaller polymers within the formulation. In aluminum-only actives and Al—Zr salts, these smaller polymers are identified as band 3 or as peak 4 (U.S. Pat. Nos. 4,359,456 and 10,526,210 describe heating aluminum active solutions). The relative concentration of these polymers is typically quantified using the band III/II ratio (Peak 4/3 ratio). Ratios exceeding 0.75 are generally indicative of a more effective active ingredient. This process, known as “activation,” results in formulations that are characterized as “activated,” “enhanced,” or “improved” antiperspirant actives. It was found that when Al—Zr salts were combined with an amino acid, and greater than 1% of an alkali metal salt to form an active premix, the active premix can have enhanced hygroscopicity and antiperspirant efficacy.

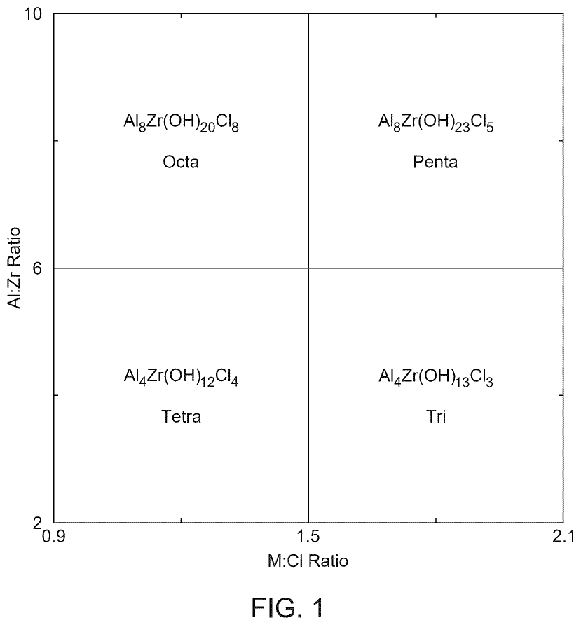

The Al—Zr salts can include “tri salts,” “tetra salts,” “penta salts,” “octa salts,” or mixtures thereof. These salts are described in Table 1, below, and FIG. 1 which shows the Al:Zr atomic ratio and the Metal:Cl atomic ratio, where the metals are aluminum and zirconium of the general formula Al_{y}Zr(OH)_{3y+4-x}Clₓ·nH₂O.

TABLE 1

| | | Aluminum to | Metal to | |
|---|---|---|---|---|
| | | Zirconium | Chloride | |
| | Common | Atomic Ratio | Atomic Ratio | |
| Compound Name | Name | (Al:Zr) | (Al + Zr):Cl | Example |
| Aluminum | Tri Salts | 2.0:1 to | 1.51:1 to | Al₄Zr(OH)₁₃Cl₃ |
| Zirconium | | 5.99:1 | 2.1:1 | |
| Trichlorohydrate | | | | |
| Aluminum | Tetra Salts | 2.0:1 to | 0.9:1 to | Al₄Zr(OH)₁₂Cl₄ |
| Zirconium | | 5.99:1 | 1.5:1 | |
| Tetrachlorohydrate | | | | |
| Aluminum | Penta Salts | 6.0:1 to | 1.51:1 to | Al₈Zr(OH)₂₃Cl₅ |
| Zirconium | | 10.0:1 | 2.1:1 | |
| Pentachlorohydrate | | | | |
| Aluminum | Octa Salts | 6.0:1 to | 0.9:1 to | Al₈Zr(OH)₂₀Cl₈ |
| Zirconium | | 10.0:1 | 1.5:1 | |
| Octachlorohydrate | | | | |

An example of a tri salt is Al₄Zr(OH)₁₃Cl₃, where the Al:Zr atomic ratio is 4 and the metal to chloride atomic ratio ((Al+Zr):Cl) is 1.67. An example of a tetra salt is Al₄Zr(OH)₁₂Cl₄, where the Al:Zr atomic ratio is 4 and the metal to chloride atomic ratio ((Al+Zr):Cl) is 1.25. An example of a penta salt is Al₈Zr(OH)₂₃Cl₅, where the Al:Zr atomic ratio is 8 and the metal to chloride atomic ratio ((Al+Zr):Cl) is 1.8. An example of an octa salt is Al₈Zr(OH)₂₀Cl₈, where the Al:Zr atomic ratio is 8 and the metal to chloride atomic ratio ((Al+Zr):Cl) is 1.125. The aluminum zirconium tri-, tetra-, penta-, and/or octa-chlorohydrate salts can be combined with an amino acid, such as glycine, and an alkali salt, such as sodium chloride. Other characteristics of Al—Zr salts can be found in the United States Pharmacopoeia and National Formulary (USP/NF) in its monograph (2024).

The active premixes can be made by combining a desired Al—Zr salt, an amino acid, an alkali metal salt, and optionally an alkaline earth metal salt in an aqueous solution at room temperature and allow to stir for 30 min to form the active premix solution. Next, the active premix solution can be dried to produce a powder, which forms the solid active premix. As an alternative approach, alkali metal salts can be incorporated after a heating process and before the drying stage to achieve the powdered form of the active premix. The particulate active premix could be generally platelet in shape and have a Dv50 average particle size of 12 to 18 microns. The active could be finely ground to have more than 85% or more than 90% (Dv90) of the particle having a size less than 10 microns and an average of less than 6 microns. Optionally, the active premix powder can also be dissolved or suspended in a solution, including aqueous, propylene glycol, propylene carbonate, or alcohol-based solution.

Each particle in the active premix can include aluminum and the alkali metal salt. The alkali metal can be interdispersed with the aluminum and/or it can be in a surface coating that can be visualized using energy-dispersive X-ray (EDX) spectroscopy coupled with a scanning electron microscopy (SEM). For instance, the alkali metal salt can be sodium chloride and each particle can contain aluminum, sodium, and chloride. The sodium can be interdispersed with the aluminum and chloride and/or the sodium can be present in a surface coating that can be visualized using EDX spectroscopy coupled with an SEM.

The active premixes containing an amino acid and alkaline earth metal, can have an amino acid to alkaline earth metal molar ratio of <5, <4, <3, ≤2, ≤1.75, ≤1.5, ≤1.4, ≤1.3, ≤1.2, or ≤1.1. The active premixes can have an amino acid to alkaline earth metal molar ratio of >0, ≥0.1, ≥0.25, ≥0.5, ≥0.75, ≥0.90, ≥1.0, ≥1.1, ≥1.25, or ≥1.3. Without wishing to be bound by theory, it is believed that lower ratios can be more effective in producing active premixes with improved hygroscopic properties. On the other hand, higher ratios are believed to be beneficial in achieving longer periods of solution stability before drying the active premix to a particle and it can be desirable to adjust the ratio based on manufacturing conditions.

The active premix can have a pH of about 3.0 to about 5, from about 3.2 to about 4.8, from about 3.4 to about 4.5, or from about 3.6 to about 4.2, as determined by the pH Test Method, described herein.

The active premix can have a maximum change in mass over 48 hours of ≥48%, ≥50%, ≥ 60%, ≥65%, ≥70%, ≥75%, ≥80%, ≥90%, ≥95%, ≥100%, ≥105%, ≥110%, ≥115%, ≥120%, ≥125%, ≥130%, or ≥140% according to the Water Vapor Sorption Test Method, described herein. In order to maintain the necessary stability and processability required for practical application in manufacturing antiperspirant formulations and shipping of antiperspirant formulations and active salts it can be advantageous for the active premix to have a maximum change in mass over 48 hours of ≤150%, ≤140%, ≤130%, ≤125%, ≤115%, or ≤100% according to the Water Vapor Sorption Test Method, described herein.

The metal to chloride atomic ratio (Al+Zr:Cl) can be from about 0.75 to about 2.5, from about 1 to about 2, from about 1.15 to about 1.7, or from about 1.17 to about 1.68. The metal to chloride atomic ratio (Al+Zr:Cl) can be ≥0.5, ≥0.7, ≥0.8, ≥0.9, ≥1, ≥1.1, or ≥1.15. The metal to chloride atomic ratio (Al+Zr:Cl) can be ≤2.25, ≤2.1, ≤2, ≤1.9, ≤1.8, or ≤1.7.

The Al:Zr atomic ratio can be from about 1 to about 15, from about 2 to about 12, or from about 3 to about 10. The Al:Zr atomic ratio can be from about 1 to about 5, from about 2 to about 4, from about 3 to about 3.5, or from about 3 to about 3.35. The Al:Zr atomic ratio can be from about 5 to about 15, from about 6 to about 12, from about 7 to about 11, from about 9 to about 10, from about 9.25 to about 9.75, or from about 9.4 to about 9.7.

The polymer size distribution of the Al—Zr in the active premix can be defined by size exclusion chromatography method using Gel Permeation Chromatography (GPC), described herein in the GPC Test Method. The active premix can have a peak IV/III (Band III/II) ratio of exceeding 0.75, ≥1, ≥1.5, ≥2, ≥2.1, ≥2.4, ≥2.5, ≥3, ≥3.25, ≥3.4, ≥3.5, ≥3.75, ≥3.85, ≥3.90, ≥4.0, ≥4.2, ≥4.4, ≥4.6, ≥4.8, ≥5.0, ≥5.2, ≥5.4, ≥5.6, ≥5.8, ≥6.0, ≥6.25, ≥6.5, ≥6.75, ≥7.0, ≥7.25, ≥7.5, ≥7.75, or ≥7.9, according to the GPC Test Method. Active premixes having a peak IV/III ratio exceeding 0.75 can be referred to as “activated” and the process of heating to increase the peak IV/III ratio can be referred to as “activation.” The peak IV/III ratio can be dependent on several factors including the level and type of alkaline earth metal salt, amount and type of amino acid, and temperature and time of heating in solution prior to drying.

Alkaline earth metal salts can include an alkaline earth metal cation and an anion that is acceptable for application to the skin. Alkaline earth metals are elements in Group 2 of the periodic table and include beryllium, magnesium, calcium, and strontium. The anions associated with the alkaline earth metals salts can include chloride, sulfate, phosphate, nitrate, or other halogen anions. More specifically, the salts can include calcium chloride, calcium nitrate, calcium sulfate, calcium phosphate, strontium chloride, strontium sulfate and strontium nitrate. Among these, calcium chloride may be preferred. The composition can include from about 0.5% to about 13%, from about 1% to about 10%, from about 1.5% to about 7%, or from about 1.75% to about 5% alkaline earth metal salts.

The amino acids can include natural amino acids, unnatural amino acids (e.g., non-canonical), and/or derivatives thereof (e.g., betaines such as trimethylglycine). The amino acids can include alanine, arginine, asparagine, aspartic acid, beta-alanine, cysteine, glutamic acid, glutamine, glycine, trimethylglycine, histidine, isoleucine, leucine, lysine, methionine, phenylalanine, proline, serine, threonine, tryptophan, tyrosine, valine, or mixtures thereof. Among these, glycine, trimethylglycine, alanine, leucine, or mixtures thereof may be preferred. Among these, glycine, alanine, and/or trimethylglycine may be preferred. The composition can include from about 5% to about 25%, from about 6% to about 17%, from about 7% to about 14%, from about 9% to about 13%, or from about 9.4% to about 11.5% of the amino acid.

It was found that active premixes containing glycine, alanine, beta-alanine, or trimethylglycine exhibited similar performance characteristics when tested in vitro. It is believed that these amino acids, along with others, may be acceptable to consumers in the active premixes described herein. Additionally, some consumers may prefer naturally derived amino acids, including trimethylglycine.

Moreover, when an alkaline earth metal is present, the active premixes can have an alkaline earth metal to aluminum atomic ratio ≥0.05, ≥0.07, ≥0.09, or ≥0.10. The active premixes can have an alkaline earth metal to aluminum atomic ratio of ≤1, ≤0.75, ≤0.5, ≤0.3, ≤0.2, ≤0.17, or 0.16. The active premixes can have an alkaline earth metal to aluminum atomic ratio of about 0.05 to about 0.5, about 0.07 to about 0.3, or about 0.1 to about 0.2. Without wishing to be bound by theory, it is believed that higher ratios can create more activation or allow activation at lower temperatures. This higher ratio may also provide longer solution stability of the polymers before the active premix is dried to a particle. However, the higher ratio can generate higher absolute levels of glycine in the active premix that may, in turn, lead to a less hygroscopic active premix.

The active premixes can also include an alkali metal salt that is acceptable for use on skin. Alkali metals are elements in group 1 of the periodic table and can include: lithium, sodium, potassium, or mixtures thereof. Anions for these salts of these metals can include chloride, sulfate, nitrate, other halogen anions, or mixtures thereof. Acceptable salts can include sodium chloride, sodium sulfate, sodium phosphate, sodium nitrate, lithium chloride, lithium sulfate, lithium nitrate, potassium chloride, potassium nitrate, potassium sulfate, potassium phosphate, or mixtures thereof. The salts can include sodium chloride, potassium chloride, lithium chloride, or mixtures thereof. Among these, sodium chloride may be preferred. Without wishing to be bound by theory, it is believed that salts can further increase the water dissolution rate of the active premix. These salts can comprise up to 25% of the active premix, less than 15%, or less than 10%. If present, these salts can comprise at least 1% of the active premix, at least 2%, at least 3%, at least 4%, or at least 5%.

The active premixes can be made according to the following method: the tri-, tetra-, penta-, or octa-salt can be dissolved in water to generate an aqueous solution. Next, the alkali metal salt can be added to the solution at room temperature and can be stirred for 1 minute up to 6 hours.

The stir time may be dependent on the concentration of Al—Zr in solutions and the amino acid to alkaline earth metal mole ratio. Stirring times, the alkaline earth metal atomic ratio, and the amino acid to alkaline earth metal mole ratio can be varied based on the desired degree of hygroscopicity and available processing equipment. If present, the alkali metal salt can be added before or after water is added.

The solution can be dried by any suitable manner including belt drying, tray drying and spray drying. Among these, spray drying may be preferred. The dried particle may be ground to desired particle size and/or sieved to desired particle distribution and may be spherical, platelet or random mixture in shape. The particle size may vary based on the final product (e.g., spray, cream, roll-on or stick product). The particulate active premix could be generally spherical in shape and can have a Dv50 average particle size of 25-35 microns. The particulate active premix could be generally platelet in shape and have a Dv50 average particle size of 12 to 18 micron. The active could be finely ground to have more than 85% or more than 90% (Dv90) of the particles having a size less than 10 microns and an average of less than 6 microns.

### Methods of Use

The particulate active premix can be incorporated into antiperspirant products. The product can be a roll-on, cream, paste, foam, or a stick including anhydrous sticks, gel sticks including aqueous gel sticks, soft solids, or glycol-based sticks. The antiperspirant product may be topically applied to the axilla or other area of the skin in any known or otherwise effective method for controlling malodor associated with perspiration. These methods comprise applying to the axilla or other area of the human skin an effective amount of the antiperspirant product that includes the active premix composition, typically about 3.5 to about 5 mg/cm² of the product, and more typically about 4 mg/cm². The product is generally a leave-on composition that can provide lasting odor protection and freshness, and the composition may provide 72 hours or more of odor control after application. The product can provide wetness protection, which can refer to the product's ability to help reduce or prevent excessive sweating and the resulting wetness under the arms.

### Method of Making

The active premix can be made according to the following method: providing one or more aqueous solutions that can include Al—Zr and/or basic aluminum chlorides and an alkali metal salt and/or magnesium chloride. The alkali metal salt, magnesium chloride, and/or amino acid can be in an aqueous solution with the Al—Zr and/or an aqueous solution with the basic aluminum chloride or a separate aqueous solution. Here are examples of aqueous solutions:
An aqueous solution that includes Al—Zr and NaCl (which is the aqueous solution premix)
An aqueous solution that includes a basic aluminum chloride and NaCl
A first aqueous solution that includes basic aluminum chloride and NaCl and a second aqueous solution that includes Al—Zr
A first aqueous solution that includes basic aluminum chloride and a second aqueous solution that includes Al—Zr and NaCl
A first aqueous solution that includes basic aluminum chloride and/or a second aqueous solution that includes Al—Zr, and a third aqueous solution that includes NaCl
A first aqueous solution that includes basic aluminum chloride and NaCl and a second aqueous solution that includes Al—Zr and NaCl

If more than one aqueous solution is provided, the solutions are mixed for a period (e.g., ≤20 minutes) to form the aqueous solution premix. The mixing may be performed by any suitable method, such as co-mixing or inline mixing.

Optionally, the aqueous solution premix can be heated along with an amino acid (if not present in the aqueous solution premix or the amino acid can be added with this step) and an alkaline earth metal salt (if present) to a temperature of from about 80° C. to about 100° C., 85° C. to about 95° C., or about 80° C. to about 90° C. to form an activated solution having the have a peak IV/III (Band III/II) ratio described herein. The heating can be performed for about 30 minutes to about 15 hours, about 1 hour to about 8 hours, about 2 hours to about 4 hours. About 2.5 hours to about 4.5 hours, or from about 2 hours to about 3 hours. Then, the activated solution is cooled to a temperature of 50° C. or below to form a cooled solution. If the cooled solution does not include Al—Zr, the cooled solution can be mixed with an Al—Zr solution.

The aqueous solution premix, the cooled solution, and/or the cooled solution mixed with an Al—Zr solution can be spray dried to isolate the corresponding powder, forming a plurality of particles of the active premix where the alkali metal and/or magnesium chloride can form a surface coating on the particles. The plurality of particles can be ground to a Dv50 particle size less than 50 microns. In some examples, water or another solvent can be added to the plurality of particles to regenerate a solution that can be incorporated into antiperspirant products.

If used, the basic aluminum chlorides have the general formula Al_{y}(OH)_{3y-z}Cl_{z}·nH₂O. Of particular interest are the basic aluminum chloride salts of formula Al₂(OH)_{3.778}Cl_{2.222} to Al₂(OH)_{5.048}Cl_{0.952}. This includes aluminum chlorohydrate (ACH), aluminum sesquichlorohydrate (ASCH), and/or aluminum dichlorohydrate (ADCH), which are detailed in Table 2 below.

TABLE 2
Basic Aluminum Chlorides

| | | Aluminum to | | |
|---|---|---|---|---|
| | | Chloride | | |
| | Abbre- | Atomic Ratio | Chemical Formula | |
| Compound Name | viation | (Al:Cl) | Range | Example |
| Aluminum | ACH | 1.91-2.10 | Al₂(OH)_{5.048}Cl_{.952} to | Al₂(OH)₅Cl |
| Chlorohydrate | | | Al₂(OH)_{4.953}Cl_{1.047} | |
| Aluminum | ASCH | 1.26-1.90 | Al₂(OH)_{4.947}Cl_{1.053} to | Al₂(OH)_{4.5}Cl_{1.5} |
| Sesquichlorohydrate | | | Al₂(OH)_{4.413}Cl_{1.587} | |
| Aluminum | ADCH | 0.90-1.25 | Al₂(OH)_{4.400}Cl_{1.600} to | Al₂(OH)₄Cl₂ |
| Dichlorohydrate | | | Al₂(OH)_{3.778}Cl_{2.222} | |

### Definitions

Except as otherwise noted, the articles “a”, “an”, and “the” mean “one or more”.

Herein, “effective” means an amount of a subject active high enough to provide a significant positive modification of the condition to be treated. An effective amount of the subject active will vary with the particular condition being treated, the severity of the condition, the duration of the treatment, the nature of concurrent treatment, and like factors.

As used herein, “visual detection” means that a human viewer can visually discern if the material had deliquesced with the unaided eye (except for standard corrective lenses adapted to compensate for near-sightedness, farsightedness, or astigmatism, or other corrected vision) in lighting at least equal to the illumination of a standard 100-watt incandescent white light bulb at 30 cm.

All numbers expressing pH values are to be understood as being modified by the term “about,” and as encompassing readings using a pH meter having a variation of up to +10%, such as up to +9%, up to +8%, up to +7%, up to +6%, up to +5%, up to +4%, up to +3%, up to +2%, or up to +1%, which a skilled person will recognize relates to the inherent variation in pH meters.

As used herein, the expressions “ranging from” and “between” are inclusive of the endpoints of the recited range(s).

All percentages are by weight of the composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 21° C. and at ambient conditions, where “ambient conditions” means conditions under about 1 atmosphere of pressure and at about 50% relative humidity. All weights as they pertain to listed ingredients are based on the active level and do not include carriers or by-products that may be included in commercially available materials, unless otherwise specified.

When a range of values is described herein, the range is contemplated to encompass the boundaries of the range as well as each value and sub-range within that range and individual values within that range. For example, “2 mg/L to 6 mg/L” is contemplated to encompass, for example, 2.0 mg/L, 2.5 mg/L, 2.7 mg/L, 3 mg/L, 4 mg/L, 4.5 mg/L, 5 mg/L, 5.5 mg/L, 6 mg/L, 2 mg/L to 3.5 mg/L, 2.5 mg/L to 4 mg/L, 2.5 mg/L to 4.5 mg/L, 4.5 mg/L to 6 mg/L, 4.8 mg/L to 5.3 mg/L, and so forth.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

### Test Methods

### pH Test Method

The pH of particulate active premixes and/or the actives are determined by creating a 15% solution of the powder in deionized water and measuring with a calibrated glass electrode and meter such as a Mettler Toledo® SevenDirect pH SD20 or equivalent. The pH of the aqueous phase is then determined.

### GPC Test Method

The GPC Test Method is performed on sample active premix to quantify relative concentration of aluminum polymer populations present. The method is preferably performed on active or active premix but can also be performed on finished antiperspirant product if active or active premix is not available. Peaks corresponding to five characteristic polymer populations are identified and integrated, and ratios of relative areas of peaks of interest are calculated and reported.

### Sample Preparation

If sample active or active premix is available, it is dissolved or dispersed in 0.01-M nitric acid such that active is present at 1.0% w/w. If only sample finished antiperspirant product is available, it is dissolved or dispersed in 0.01-M nitric acid such that the active is present at 1.0% w/w. After dilution, the nitric acid solution may be filtered (for example, using a 0.45-μm nylon syringe filter) if necessary. In any case, only the resulting nitric-acid extraction solution is subsequently injected into the chromatograph.

### Apparatus and Procedure

GPC chromatographic separation is performed using 0.01-M nitric acid mobile phase, using a total 900 mm length of silica columns. Exemplary suitable apparatus consists of three consecutive μPorasil Columns, 3.9×300 mm, 10 mm packing (available from Waters, Milford, Massachusetts), plumbed in series. The chromatograph is equipped with a refractive-index detector. Exemplary suitable refractive-index detector is ERC RefractoMax 520 (available from IDEX, Oak Harbor, Washington). A 5-μL injection volume is used, and an exemplary suitable flow rate is 0.8 mL/min.

### Analysis

Chromatograms are processed and peaks integrated, for example using suitable software such as Thermo Fisher Scientific Chromeleon Data System. The focus of this analysis is a set of four or five peaks appearing early in the chromatogram that correspond to five characteristic polymer populations. (Sharper peaks, generally with longer retention time, are understood to correspond to small molecules present in the extracted active and are disregarded in this analysis.) The peaks observed in the chromatogram are designated in order of appearance on the chromatogram as Peaks I-II (generally appears as a single peak) and Peaks III, IV and V. A representative chromatogram containing the abovementioned peaks is shown in FIG. 6. The area of Peaks I-II, III, IV and V correspond to the relative concentration of aluminum polymer populations present in the analyzed active in the injected sample. (It is noted that these five polymer populations are generally recognized as present. Swaile U.S. Pat. No. 6,149,897 refers to the peaks in the chromatograms as Peaks I-II, Peak III, Peak IV, and Peak V. Similarly, Gosling U.S. Pat. No. 4,359,456 refers to Peak I-II as “Band 1,” Peak III as “Band 2, Peak IV as “Band 3”, and Peak V as “Band 4.”

Peaks I-II, III, IV, and V are each integrated, and their peak areas (arbitrary units) are recorded. The ratios between combinations of peaks of interest may be calculated and expressed as a dimensionless ratio. For example, the ratio of the area of Peak IV to the area of Peak III is calculated and reported as the “Peak IV/III ratio” to the nearest 0.01.

### Water Vapor Sorption Test Method

The Water Vapor Sorption Test Method is used to quantify the amount of water vapor uptake by a sample or composition when contained in a warm, moist environment. This method makes use of a dynamic vapor sorption (DVS) instrument to first dry and condition a specimen of a sample active premix or composition and then measure the increase of mass over time as the sample is held at elevated temperature and relative humidity.

A DVS instrument suitable for this method is capable of controlling percent relative humidity (% RH) to within ±5% RH, temperature to within ±2° C., and measuring mass to a precision of ±0.01 mg. It is further capable of maintaining environments of 0% RH at both 37° C. and 50° C. and 90% RH at 37° C. One suitable exemplary apparatus is the S Endeavour Dynamic Vapor Sorption Analyzer (Surface Measurement Systems Ltd.), or equivalent. The DVS instrument is configured to record the mass of specimen at ten-minute intervals with a precision of 0.01 mg or better.

A 20.0±5.0 mg specimen of material is spread evenly on a tared aluminum sample pan appropriate for the DVS instrument. The specimen and pan are introduced into the DVS, and the specimen is first dried at 37° C. and 0% RH for 10 hours. The specimen is then conditioned at 37° C. and 0% RH for 20 hours. The specimen mass point recorded at 37° C. and 0% RH is taken as the initial specimen mass, denoted mᵢ. The specimen is then exposed to an environmental state at 37° C. and 90% RH for 18 hours. For any point in time/during the final 18 hours of the measurement, the percent change in mass of the specimen is calculated by the expression:

$$Percent change in mass [ \% ] = \frac{{m}_{t} - {m}_{i}}{{m}_{i}} \times 100 \%$$

where mₜ is the measured specimen mass at that point in time, and time t is the total time passed after the beginning of the first, drying condition of the DVS. The maximum percent change in mass of the specimen observed over the final 18 hours of the measurement, corresponding to time 30 hours to 48 hours of the DVS measurement procedure overall, is reported as the Change in Mass of the specimen, reported in percent to the nearest 1%.

### Examples

The active premix (APM) examples in Table 3 to Table 4 were prepared using the following method: The appropriate amount of AP active, sodium chloride, and water was added to a beaker equipped with a stirbar. The mixture was stirred for a minimum of 1 minute and up to 6 hours at room temperature, during which time it became homogeneous. Following this, the resulting solution was spray dried to isolate the corresponding powder. Spray drying was conducted using a Büchi Mini Spray Dryer S-300 (Model: Corrosives 250° C. Pro) using the following conditions: Drying gas 30.5 m³/h, Spray gas 1200 L/h, Inlet temperature: 220° C., Outlet temperature 116° C., once spray drying was completed, the APM was collected in a vessel for further analysis using various analytical methods.

The pH for the examples in Table 3 to Table 4 was determined according to the pH Test Method, described herein.

The Kinetic Study for the examples in Table 3 to Table 4 was performed according to the Water Vapor Sorption Test Method, described herein. The test determined the amount of water vapor sorption that occurs in a raw material.

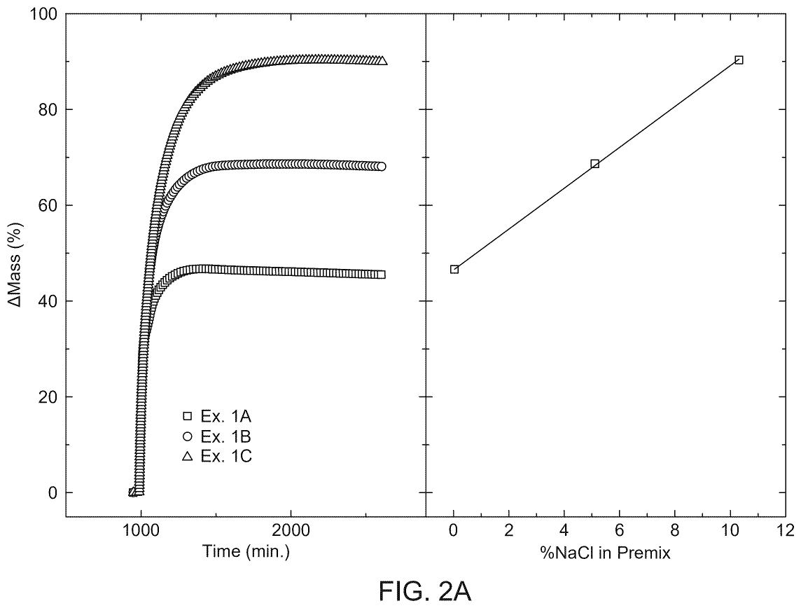

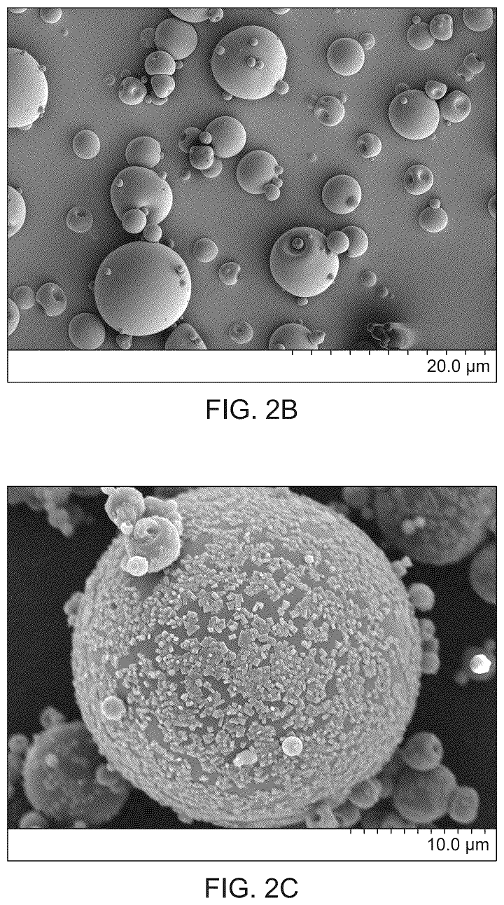

FIGS. 2A, 3A, 4A, and 5A show that as the amount of sodium chloride added increases, the hygroscopicity also increases. These figures also surprisingly show that there is a linear dose response based on the amount of sodium chloride that is added. This indicates that sodium chloride enhances the moisture-absorbing properties of the active premix compared to formulations without sodium chloride, as demonstrated in APM Examples 1A, 2A, 3A, and 4A. The increased ability to absorb moisture from the environment due to the added sodium chloride is expected to facilitate a faster dissolution of the active premix in sweat, ultimately enhancing the effectiveness of the antiperspirant.

Additionally, a direct comparison of Examples 1B vs. 1C, 2B vs. 2C, 3B vs. 3C, and 4B vs. 4C illustrates that higher sodium chloride content correlates with greater hygroscopicity. However, it is important to recognize that while increased hygroscopicity can enhance the performance of the active premix, there is a trade-off. If the active premix becomes too hygroscopic, it can create challenges in shipping and incorporating it into antiperspirant formulations. It was observed through visual detection that when an active premix containing aluminum zirconium octachlorohydrate salt, glycine, and sodium chloride absorbed more than 125% moisture over a 48-hour period, the active premix began to deliquesce within 30 minutes under ambient conditions.

The SEM images in FIGS. 2B-2C, 3B-3C, 4B-4C, and 5B-5C clearly show the effects of adding sodium chloride to the system. When no sodium chloride is added, the particles appear relatively smooth, as seen in FIGS. 2B, 3B, 4B, and 5B. However, after the addition of sodium chloride, the particles exhibit a rougher surface, with visible cubic sodium chloride crystals present, as shown in FIGS. 2C, 3C, 4C, and 5C.

It was surprising to find sodium chloride deposits on the particle surfaces, especially since the spray-dried mixture is homogeneous and contains relatively low sodium compared to the amounts of aluminum and zirconium (with a Na to Al+Zr atomic ratio of 0.12-0.14). In some instances, the sodium chloride covers a significant portion of the particle surface (e.g., more than a majority, >80% up to 95%) while in other cases, only about 50% may be covered, as shown in the SEM images.

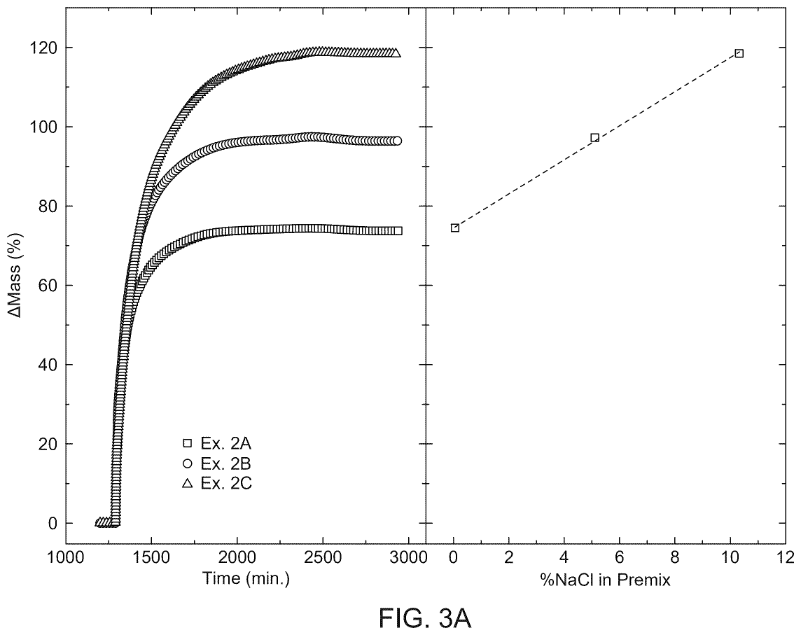

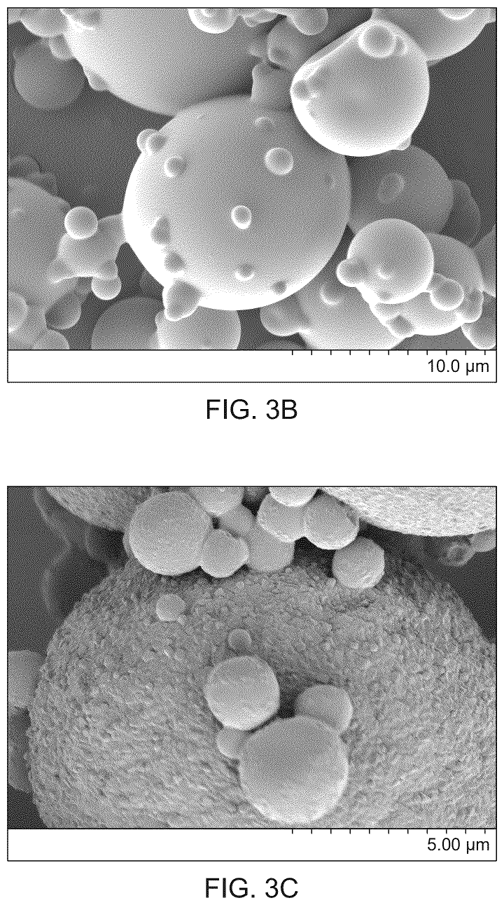

TABLE 3

| | Ex. 1A | Ex. 1B | Ex. 1C | Ex. 2A | Ex. 2B | Ex. 2C |
|---|---|---|---|---|---|---|
| | APM | APM | APM | APM | APM | APM |
| % Al | 14.77 | 13.66 | 13.20 | 13.7 | 13.10 | 12.13 |
| % Zr | 15.3 | 14.5 | 13.9 | 15.1 | 13.7 | 12.9 |
| % Cl (Total) | 15.83 | 17.83 | 20.33 | 20.25 | 22.12 | 23.96 |
| % Glycine | 12.97 | 14.42 | 12.06 | 12.75 | 12.54 | 11.52 |
| (measured) | | | | | | |
| % Glycine | 14.97 | 14.22 | 13.47 | 12.59 | 11.96 | 11.33 |
| Target | | | | | | |
| Al:Zr Atomic | 3.33 | 3.25 | 3.27 | 3.13 | 3.27 | 3.43 |
| Ratio | | | | | | |
| Al—Zr:Cl | 1.59 | 1.59 | 1.61 | 1.18 | 1.17 | 1.15 |
| Atomic Ratio | | | | | | |
| % Na | 0.0258 | 2.01 | 4.06 | 0.0211 | 2.00 | 3.94 |
| Target NaCl | 0 | 5 | 10 | 0 | 5 | 10 |
| Calculated | 0.066 | 5.11 | 10.32 | 0.05 | 5.08 | 10.02 |
| NaCl | | | | | | |
| Active pH | 3.92 | 4.12 | 4.1 | 3.63 | 3.65 | 3.64 |
| GPC Peak 4:3 | 0.66 | 0.59 | 0.85 | 0.76 | 0.81 | 0.85 |
| Ratio | | | | | | |
| Kinetic Study | FIG. 2A | | | FIG. 3A | | |
| SEM Image | FIG. 2B | FIG. 2C | | FIG. 3B | FIG. 3C | |

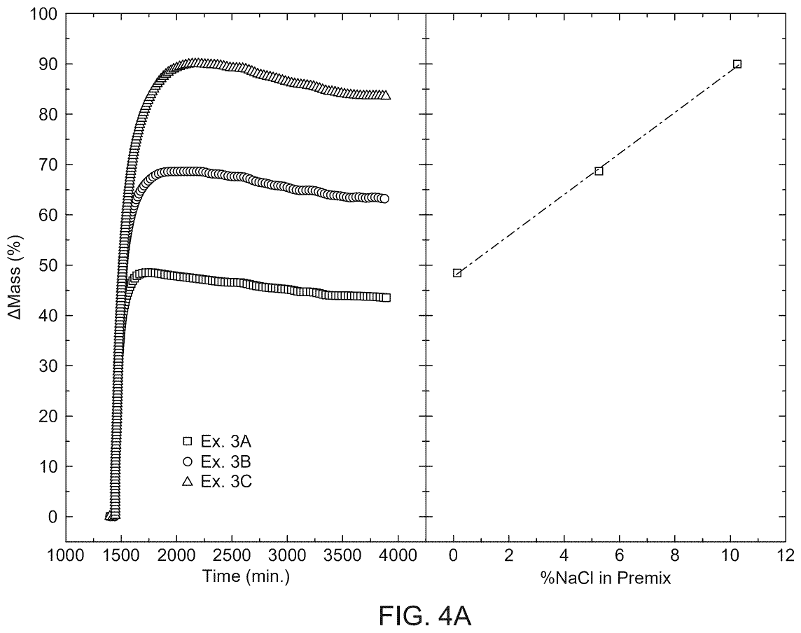

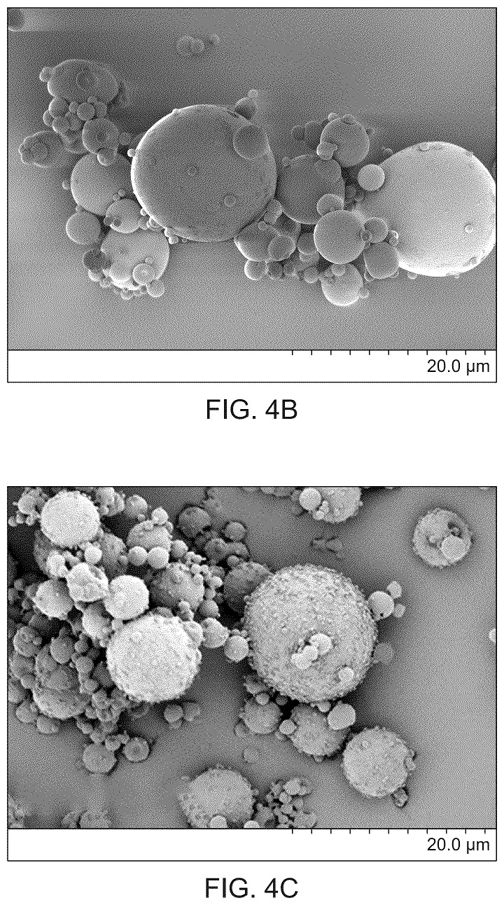

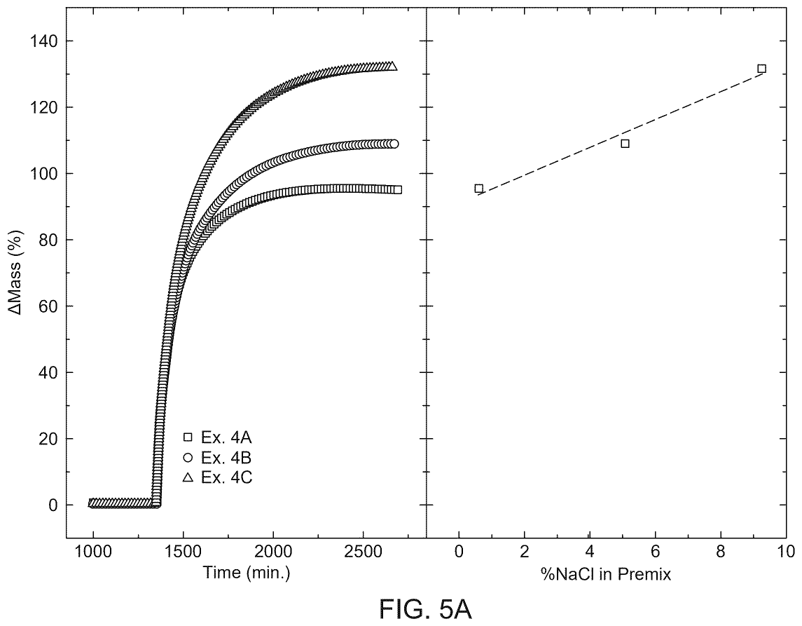

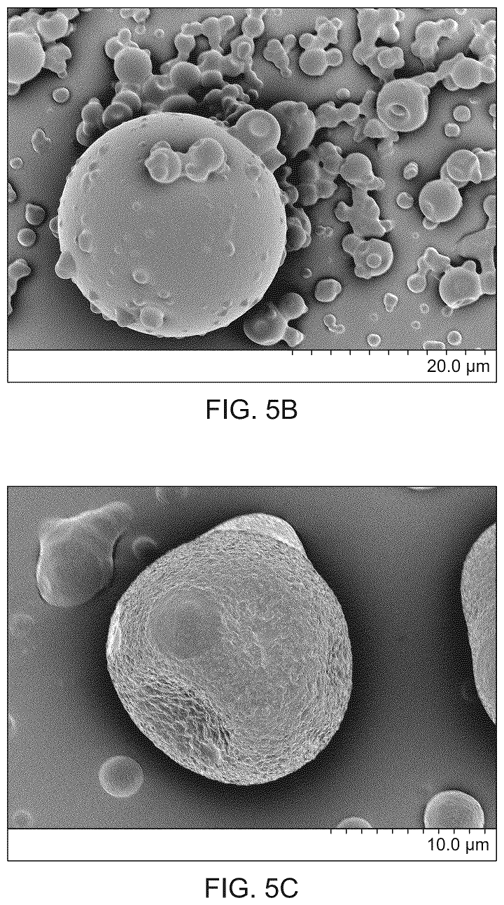

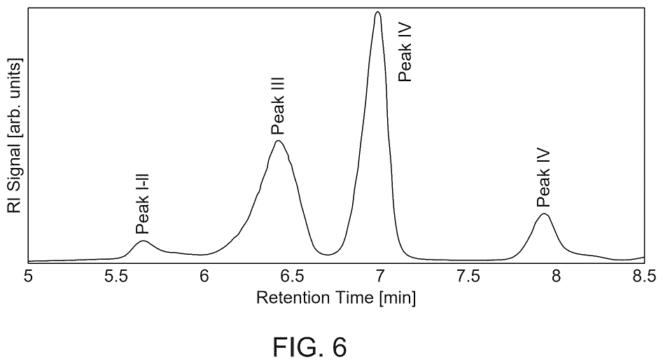

TABLE 4

| | Ex. 4A | Ex. 4B | Ex. 4C | Ex. 5A | Ex. 5B | Ex. 5C |
|---|---|---|---|---|---|---|
| | APM | APM | APM | APM | APM | APM |
| % Al | 20.1 | 19.1 | 17.9 | 16.45 | 16 | 15.42 |
| % Zr | 7.22 | 6.87 | 6.42 | 5.88 | 5.80 | 5.59 |
| % Cl (Total) | 17.45 | 19.65 | 21.79 | 22.65 | 24.89 | 26.38 |
| % Ca | 0 | 0 | 0 | 3.82 | 3.73 | 3.6 |
| % Glycine | 10.40 | 8.63 | 7.29 | 9.06 | 9.04 | 8.18 |
| (measured) | | | | | | |
| % Glycine | 9.80 | 9.31 | 8.82 | 8.81 | 8.63 | 8.46 |
| Target | | | | | | |
| Al:Zr Molar | 9.59 | 9.58 | 9.61 | 9.64 | 9.51 | 9.51 |
| Ratio | | | | | | |
| Al—Zr:Cl | 1.68 | 1.68 | 1.67 | 1.20 | 1.19 | 1.22 |
| Atomic Ratio | | | | | | |
| % Na | 0.0503 | 2.07 | 4.04 | 0.24 | 2 | 3.64 |
| Target % NaCl | 0 | 5 | 10 | 0 | 5 | 10 |
| Calculated | 0.13 | 5.26 | 10.27 | 0.61 | 5.08 | 9.25 |
| % NaCl | | | | | | |
| Active pH | 4.15 | 4.17 | 4.16 | 3.94 | 4.08 | 4.18 |
| GPC Peak 4:3 | 0.75 | 0.69 | 0.78 | 1.72 | 1.81 | 1.81 |
| Ratio | | | | | | |
| Kinetic Study | FIG. 4A | | | FIG. 5A | | |
| SEM Image | FIG. 4B | FIG. 4C | | FIG. 5B | FIG. 5C | |

Table 5 shows the maximum moisture pickup over 48 hours as determined by the Water Vapor Sorption Test Method, described hereafter. It was found that the active premix examples with 10% sodium chloride concentration (e.g., Ex. 1C, 2C, 3C, and 4C) had better maximum moisture pickup as compared to the examples with 5% sodium chloride (e.g., Ex. 1B, 2B, 3B, and 4B) or no sodium chloride (e.g., Ex. 1A, 2A, 3A, and 4A).

TABLE 5

| | Target Added Sodium | Maximum Moisture Pickup |
|---|---|---|
| | Chloride Concentration (%) | over 48 hours (%) |
| Ex. 1A APM | 0 | 47 |
| Ex. 1B APM | 5 | 69 |
| Ex. 1C APM | 10 | 90 |
| Ex. 2A APM | 0 | 74 |
| Ex. 2B APM | 5 | 97 |
| Ex. 2C APM | 10 | 118 |
| Ex. 3A APM | 0 | 48 |
| Ex. 3B APM | 5 | 69 |
| Ex. 3C APM | 10 | 90 |
| Ex. 4A APM | 0 | 95 |
| Ex. 4B APM | 5 | 109 |
| Ex. 4C APM | 10 | 132 |

### Combinations:

A. A particulate active premix composition comprising:
   a. an aluminum zirconium salt chosen from aluminum zirconium trichlorohydrate salt, aluminum zirconium tetrachlorohydrate salt, aluminum zirconium pentachlorohydrate salt, aluminum zirconium octachlorohydrate salt, or mixtures thereof;
   b. an amino acid;
   c. greater than 1% of an alkali metal salt and/or magnesium chloride; wherein the composition comprises a plurality of particles or a solution.
B. The composition according to Paragraph A, wherein the composition comprises up to 25% of the alkali metal salt, preferably up to 20% alkali metal salt, more preferably up to 15% alkali metal salt, and most preferably up to 10% alkali metal salt.
C. The composition according to Paragraphs A-B, wherein the composition comprises an alkali metal salt chosen from sodium chloride, potassium chloride, lithium chloride, or mixtures thereof.
D. The composition according to Paragraphs A-C, wherein the alkali metal salt comprises sodium chloride.
E. The composition according to Paragraphs C-D, wherein the aluminum zirconium salt comprises aluminum zirconium trichlorohydrate having an Al:Zr atomic ratio of from about 2.0:1 to about 5.99:1 and a metal to chloride atomic ratio (Al+Zr:Cl) of about 1.51:1 to about 2.10:1.
F. The composition according to Paragraphs C-E, wherein the aluminum zirconium salt comprises aluminum zirconium tetrachlorohydrate having an Al:Zr atomic ratio of from about 2.0:1 to about 5.99:1 and a metal to chloride atomic ratio (Al+Zr:Cl) of about 0.9:1 to about 1.50:1.
G. The composition according to Paragraph C—F, wherein the aluminum zirconium salt comprises aluminum zirconium pentachlorohydrate having an Al:Zr atomic ratio of from about 6.0:1 to about 10.0:1 and a metal to chloride atomic ratio (Al+Zr:Cl) of about 1.51:1 to about 2.10:1.
H. The composition according to Paragraph C-G, wherein the aluminum zirconium salt comprises aluminum zirconium octachlorohydrate having an Al:Zr atomic ratio of from about 6.0:1 to about 10.0:1 and a metal to chloride atomic ratio (Al+Zr:Cl) of about 0.9:1 to about 1.5:1.
I. The composition according to Paragraphs A-H, wherein the composition comprises from about 5% to about 25% of the amino acid, preferably from about 6% to about 17% of the amino acid, more preferably from about 7% to about 14% of the amino acid, and even more preferably about 9% to about 13% of the amino acid.
J. The composition according to Paragraphs A-I, wherein the amino acid is chosen from alanine, arginine, asparagine, aspartic acid, beta-alanine, cysteine, glutamic acid, glutamine, glycine, trimethylglycine, histidine, isoleucine, leucine, lysine, methionine, phenylalanine, proline, serine, threonine, tryptophan, tyrosine, valine, or mixtures thereof. Among these, glycine, trimethylglycine, alanine, leucine, or mixtures thereof.
K. The composition according to Paragraphs A-J, wherein the amino acid is chosen from glycine, alanine, trimethylglycine, or mixtures thereof.
L. The composition according to Paragraphs A-K, wherein the amino acid is chosen from glycine, alanine, beta-alanine, trimethylglycine, or mixtures thereof.
M. The composition according to Paragraphs A-L, wherein the amino acid is glycine.
N. The composition according to Paragraphs A-M, wherein the composition further comprises an alkaline earth metal salt.
O. The composition according to Paragraph N, wherein the alkaline earth metal salt is chosen from calcium chloride, calcium nitrate, calcium sulfate, strontium chloride, strontium sulfate, strontium nitrate, or mixtures thereof.
P. The composition according to Paragraphs N—O, wherein the alkaline earth metal salt comprises calcium chloride.
Q. The composition according to Paragraphs N—P, wherein the amino acid to alkaline earth metal molar ratio is <5, preferably <3, more preferably ≤1.5, and even more preferably ≤1.3.
R. The composition according to Paragraphs N-Q, wherein the amino acid to alkaline earth metal molar ratio is >0, preferably ≥0.25, more preferably ≥0.5, even more preferably ≥1.0, and even more preferably ≥1.2.
S. The composition according to Paragraphs A-R, wherein the composition has a pH of about 3.0 to about 5, preferably from about 3.2 to about 4.8, more preferably from about 3.4 to about 4.5, and even more preferably from about 3.6 to about 4.2, as determined by the pH Test Method.
T. The composition according to Paragraphs A-S, wherein the composition has a maximum change in mass over 48 hours of ≥48%, preferably ≥60%, more preferably ≥70%, even more preferably ≥80%, and most preferably ≥90% according to the Water Vapor Sorption Test Method, described herein.
U. The composition according to Paragraphs A-T, wherein the composition has a maximum change in mass over 48 hours of ≤150%, preferably ≤140%, more preferably ≤125%, and even more preferably ≤115%, according to the Water Vapor Sorption Test Method, described herein.
V. The composition according to Paragraphs C—U, wherein the composition comprises a metal to chloride atomic ratio (Al+Zr:Cl) of ≥0.5, preferably ≥0.7, more preferably ≥0.8, even more preferably ≥1, and most preferably ≥1.1.
W. The composition according to Paragraphs C—V, wherein the composition comprises a metal to chloride atomic ratio (Al+Zr:Cl) of ≤2.25, preferably ≤2.1, more preferably ≤1.9, even more preferably ≤1.8, and most preferably ≤1.7.
X. The composition according to Paragraphs C—U, wherein the composition comprises a metal to chloride atomic ratio (Al+Zr:Cl) from about 0.75 to about 2.5, preferably from about 1 to about 2, more preferably from about 1.15 to about 1.7, and even more preferably from about 1.17 to about 1.68.
Y. The composition according to Paragraph D, wherein each particle comprises aluminum, chloride, and sodium.
Z. The composition according to Paragraph Y, wherein each particle further comprises a surface coating and at least a portion of the sodium is present in the surface coating, preferably >50% of the sodium is present in the surface coating, more preferably >75% of the sodium is present in the surface coating.
AA. The composition according to Paragraphs Y—Z, wherein at least a portion of the sodium is interdispersed with the aluminum and chloride.
BB. The composition according to Paragraph Y, wherein the particle is homogeneous.
CC. The composition of according to Paragraphs A-BB, wherein the composition has a peak IV/III ratio of exceeding 0.75, preferably greater than or equal to 1, preferably greater than or equal to 1.5, preferably greater than or equal to 2, preferably greater than or equal to 2.5, preferably greater than or equal to 3, preferably greater than or equal to 3.25, preferably greater than or equal to 3.5, preferably greater than or equal to 3.75, and preferably greater than or equal to 3.85, according to the GPC Method.
DD. The composition of according to Paragraphs A-CC, wherein the composition has a peak IV/III ratio of from about 0.75 to about 7.9, preferably from about 1.5 to about 5, more preferably from about 2 to about 4.5, more preferably from about 2.1 to about 4.25, more preferably from about 2.15 to about 4.1, even more preferably from about 2.25 to about 4, and even more preferably from about 2.4 to about 4.
EE. The composition of according to Paragraphs A-DD, wherein the composition has a peak IV/III ratio of from about 2 to about 4, preferably from about 2.1 to about 3.75, more preferably from about 2.2 to about 3.5.
FF. An antiperspirant product, wherein the product comprises the composition according to Paragraphs A-EE, wherein the antiperspirant product is in the form of a roll-on, cream, paste, foam, wipes, or stick, preferably a stick.
GG. A method of making a particulate antiperspirant active premix according to Paragraphs A-EE comprising:
   a. providing an aqueous solution of an aluminum zirconium salt with a USP assay from about 10% to about 50%; wherein the aluminum zirconium salt is chosen from aluminum zirconium trichlorohydrate salt, aluminum zirconium tetrachlorohydrate salt, aluminum zirconium pentachlorohydrate salt, aluminum zirconium octachlorohydrate salt, or mixtures thereof;
   b. adding an alkali metal salt at a concentration of more than 0.5%, by weight of the aqueous solution, and an amino acid to form the aqueous solution premix;
   c. after step (b), drying, preferably spray drying, the aqueous premix solution to a plurality of particles, forming the particulate antiperspirant active premix; and
   d. optionally grinding the particulate antiperspirant active premix to a Dv50 particle size less than 50 microns.
HH. The method of Paragraph FF, further comprising adding calcium chloride to the aqueous solution in step (b); wherein the calcium to aluminum atomic ratio of about 0.05 and adding glycine to provide a glycine to calcium molar ratio from about 1 to about 2.
II. A method of making a particulate antiperspirant active premix according to Paragraphs A-EE comprising:
   a. providing an aqueous solution premix comprising an aluminum zirconium salt, an alkali metal salt and/or magnesium chloride, an amino acid, and optionally an alkaline earth salt;
   b. drying, preferably spray drying, the aqueous premix solution to a plurality of particles, forming the particulate antiperspirant active premix wherein the alkali metal salt and/or the magnesium chloride form a surface coating on the particles;
   c. optionally grinding the particulate antiperspirant active premix to a Dv50 particle size less than 50 microns;
   d. optionally adding water to regenerate a solution comprising the aluminum zirconium salt, an alkali metal salt, and/or magnesium chloride.
JJ. The method according to Paragraphs II, further comprising before drying, heating the aqueous solution premix at a temperature of from 80° C. to 100° C., preferably from about 80° C. to about 95° C., and more preferably from about 85° C. to about 90° C. to form an activated solution having a peak IV/III ratio of from about 0.75 to about 7.9, preferably from about 1.5 to about 5, and more preferably from about 2 to about 4.5.
KK. The method according to Paragraph JJ, wherein the heating is performed for about 30 minutes to about 15 hours, preferably from about 1 hour to about 8 hours, and even more preferably from about 2 hours to about 4 hours.
LL. The method according to Paragraph JJ, wherein the activated solution is cooled to a temperature of 50° C. to form a cooled solution and then drying the cooled solution.
MM. The method according to Paragraphs II-LL, wherein the aqueous solution premix further comprises a basic aluminum chloride having according of formula Al₂(OH)_{3.778}Cl_{2.222} to Al₂(OH)_{5.048}Cl_{0.952}.
NN. A method of making a particulate antiperspirant active premix according to Paragraphs A-EE comprising:
   a. providing an aqueous solution premix comprising a basic aluminum chloride having according of formula Al₂(OH)_{3.778}Cl_{2.222} to Al₂(OH)_{5.048}Cl_{0.952}, an alkali metal salt and/or magnesium chloride, an amino acid, and optionally an alkaline earth metal salt;
   b. heating the aqueous solution premix at a temperature of from 80° C. to 100° C., preferably from about 80° C. to about 95° C., and more preferably from about 85° C. to about 90° C. to form an activated solution having a peak IV/III ratio of from about 1.5 to about 7.9, preferably from about 1.75 to about 5, and more preferably from about 2 to about 4.5;
   c. optionally cooling the activated solution to a temperature of 50° C.;
   d. providing and mixing an aqueous solution of a zirconium salt to the activated solution to create an aluminum/zirconium salt premix;
   e. drying, preferably spray drying, the cooled solution to a plurality of particles, forming the particulate antiperspirant active premix wherein the alkali metal salt and/or the magnesium chloride form a surface coating on the particles;
   f. optionally grinding the particulate antiperspirant active premix to a Dv50 particle size less than 50 microns.
OO. The method according to Paragraph NN, wherein the heating is performed for about 30 minutes to about 15 hours, preferably from about 1 hour to about 8 hours, and even more preferably from about 2 hours to about 4 hours.
PP. A non-therapeutic method of reducing perspiration from the surface of the human body, comprising a step of topical application of a product comprising the particulate active premix composition according to Paragraphs A-EE.
QQ. The method of Paragraph PP, wherein said composition is applied to the underarms.
RR. A non-therapeutic use of an antiperspirant product comprising the particulate active premix composition according to Paragraphs A to EE for reduction of bodily perspiration.
SS. Non-therapeutic cosmetic use of the particulate active premix composition according to Paragraphs A to EE as an antiperspirant agent.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A particulate active premix composition comprising:
a. an aluminum zirconium salt chosen from aluminum zirconium trichlorohydrate salt, aluminum zirconium tetrachlorohydrate salt, aluminum zirconium pentachlorohydrate salt, aluminum zirconium octachlorohydrate salt, or mixtures thereof;
b. an amino acid;
c. greater than 1% of an alkali metal salt and/or magnesium chloride; comprises a plurality of particles or a solution.

2. The composition of claim 1, wherein the composition comprises up to 25% of the alkali metal salt.

3. The composition of claim 2, wherein the composition comprises an alkali metal chosen from sodium chloride, potassium chloride, lithium chloride, or mixtures thereof.

4. The composition of claim 3, wherein the composition comprises a metal to chloride atomic ratio (Al+Zr:Cl) of ≥0.5.

5. The composition of claim 3, wherein the alkali metal salt comprises sodium chloride and wherein each particle comprises aluminum, chlorine, and sodium.

6. The composition according to claim 5, wherein each particle further comprises a surface coating and at least a portion of the sodium is present in the surface coating.

7. The composition according to claim 6, wherein at least a portion of the sodium is interdispersed with the aluminum and chloride.

8. The composition of claim 1, wherein the composition comprises from about 5% to about 25% of the amino acid.

9. The composition of claim 1, wherein the amino acid is chosen from alanine, arginine, asparagine, aspartic acid, beta-alanine, cysteine, glutamic acid, glutamine, glycine, trimethylglycine, histidine, isoleucine, leucine, lysine, methionine, phenylalanine, proline, serine, threonine, tryptophan, tyrosine, valine, or mixtures thereof.

10. The composition of claim 1, wherein the composition comprises a pH of from about 3.0 to about 5.

11. The composition of claim 1, wherein the composition has a maximum change in mass over 48 hours of greater than or equal to 48% according to the Water Vapor Sorption Test Method.

12. An antiperspirant product, wherein the product comprises the composition of claim 1, wherein the antiperspirant product is in the form of a roll-on, cream, paste, foam, wipes, or stick.

13. A particulate active premix composition comprising:
a. an aluminum zirconium salt chosen from aluminum zirconium trichlorohydrate salt, aluminum zirconium salt, tetrachlorohydrate aluminum zirconium pentachlorohydrate salt, aluminum zirconium octachlorohydrate salt, or mixtures thereof;
b. an amino acid comprising glycine;
c. greater than 1% of an alkali metal salt comprising sodium chloride; comprises a plurality of particles or a solution.

14. The composition according to claim 13, wherein each particle further comprises a surface coating and at least a portion of the sodium is present in the surface coating.

15. The composition of claim 13, wherein the composition further comprises an alkaline earth metal salt.

16. The composition of claim 15, wherein the alkaline earth metal salt is chosen from calcium chloride, calcium nitrate, calcium sulfate, calcium glycinate, strontium chloride, strontium sulfate, and strontium nitrate, or mixtures thereof.

17. The composition of claim 16, wherein the alkaline earth metal salt comprises calcium chloride.

18. A method of making a particulate antiperspirant active premix according comprising:
a. providing an aqueous solution premix comprising a basic aluminum chloride having according of formula Al₂(OH)_{3.778}Cl_{2.222} to Al₂(OH)_{5.048}Cl_{0.952}, an alkali metal salt and/or magnesium chloride, and an amino acid;
b. heating the aqueous solution premix at a temperature of from 80° C. to 100° C. to form an activated solution having a peak IV/III ratio of from about 0.75 to about 7.9;
c. optionally cooling the activated solution to a temperature of 50° C.;
d. providing and mixing an aqueous solution of a zirconium salt to the activated solution to create an aluminum/zirconium salt premix;
e. drying the aluminum/zirconium salt premix to a plurality of particles, forming the particulate antiperspirant active premix wherein the alkali metal salt and/or the magnesium chloride form a surface coating on the particles.

19. The method of claim 18, wherein the drying is spray drying.

20. The method of claim 18, further comprising adding calcium chloride to the aqueous solution premix in step (a); wherein the amino acid is glycine; wherein the solution has a calcium to aluminum atomic ratio of about 0.05 and a glycine to calcium molar ratio from about 1 to about 2.
